(19) United States
(12) Patent Application Publication
(10) Pub. No.: US 2022/0233117 A1
(43) Pub. Date: Jul. 28, 2022

(54) BLOOD SAMPLING DEVICE, SYSTEM, AND METHOD

(71) Applicant: PROBUS MEDICAL TECHNOLOGIES INC., Redmond, WA (US)

(72) Inventors: Kyonghoon LEE, Redmond, WA (US); Hankil BOO, Redmond, WA (US)

(21) Appl. No.: 17/609,660

(22) PCT Filed: May 14, 2020
(86) PCT No.: PCT/US20/32980

§ 371 Date: Nov. 8, 2021

Related U.S. Application Data
(60) Provisional application No. 62/848,426, filed on May 15, 2019.
(60) Provisional application No. 62/849,691, filed on May 17, 2019.
(60) Provisional application No. 62/977,044, filed on Feb. 14, 2020.

(51) Int. Cl. A61B 5/15; A61B 5/151

(52) U.S. Cl. CPC A61B 5/150022; A61B 5/15186; A61B 5/150389; A61B 5/150358

(57) ABSTRACT

Provided is a blood sampler including a needle for piercing a user's skin and a sampler body for receiving and holding a test strip in place during blood sampling and testing. When the test strip is inserted into the sampler body, the sampler body aligns one end of the test strip with the needle such that the blood sampled through the needle ends up on the test strip, while keeping the other end of the test strip exposed outside the sampler body such that a blood sample analyzer can be connected to the test strip without removing the test strip from the sampler body. The blood sampler and the test strip collectively form a cartridge, which can be inserted into a launcher for launching the blood sampling cartridge towards the user's skin to facilitate blood sampling.

### BACKGROUND

A high level of interest exists in the healthcare community and industry for improving the technologies of sensing and monitoring glucose levels. Today, most glucose sensors use an electrochemical method.

### SUMMARY

The present disclosure provides a blood sampler that includes a needle for piercing a user's skin and a sampler body for receiving a test strip and holding the test strip in place during the blood sampling and testing. When the test strip is inserted into the sampler body of the blood sampler, the sampler body aligns one end of the test strip with the needle such that the blood sampled through the needle ends up on the test strip while keeping the other end of the test strip exposed outside the sampler body such that a blood sample analyzer can be connected to the test strip without having to remove the test strip from the sampler body. In the present disclosure, the blood sampler and the test strip are collectively referred to as a blood sampling cartridge.

The present disclosure also provides a blood sampling cartridge launcher. The launcher includes an opening for receiving the blood sampling cartridge and a launching mechanism for launching the blood sampling cartridge towards the user's skin to sample the user's blood. When the blood sampling cartridge is inserted into the launcher and launched, the needle on the blood sampling cartridge pierces the user's skin, and blood is collected onto the test strip through the needle. The launcher may include a hinge, which allows the launcher to be in one of at least two hinge positions. In one hinge position, the blood sampling cartridge is aligned with the launching mechanism and the test strip is enclosed inside the launcher. This first hinge position allows the test strip to be protected inside the launcher, and allows the blood sampling cartridge to be launched for sampling the user's blood. In another hinge position, the blood sampling cartridge is not aligned with the launching mechanism and the test strip is exposed outside the opening of the launcher. This second hinge position allows the blood sampling cartridge to be inserted into or removed from the launcher, and allows a blood sample analyzer to be connected to the test strip while the blood sampling cartridge is still inserted into the launcher.

### BRIEF DESCRIPTION OF THE DRAWINGS

The foregoing and other features, aspects and advantages of the present invention are described in detail below with reference to the drawings of various implementations, which are intended to illustrate and not to limit the invention. The drawings comprise the following figures in which:

FIG. 1A illustrates a configuration of a blood sampling cartridge and a blood sampling cartridge launcher, according to an example implementation;

FIG. 1B illustrates a configuration of a blood sampling cartridge launcher accommodating a blood sampling cartridge therein, according to an example implementation;

FIG. 2A illustrates an example in which a user holding a cartridge launcher against the user's finger;

FIG. 2B illustrates an example in which the user of FIG. 2A has actuated the launching mechanism with the user's thumb, causing the blood sampling cartridge inside the launcher to move towards the user's finger;

FIG. 3A illustrates the components of a blood sampling cartridge and a blood sampling cartridge launcher, according to an example implementation;

FIG. 3B illustrates an example of a needle, according to an example implementation;

FIG. 4A illustrates components of a blood sampling cartridge, according to an example implementation;

FIG. 4B illustrates a needle and a surrounding component in a blood sampling cartridge, according to an example implementation;

FIG. 5 illustrates a flowchart depicting a method for making the blood sampling cartridge, according to an example implementation;

FIG. 6A illustrates an example of relative positions of the needle and the sampler body, according to example implementations;

FIG. 6B illustrates another example of relative positions of the needle and the sampler body, according to an example implementation;

FIG. 6C illustrates another example of relative positions of the needle and the sampler body, according to an example implementation;

FIG. 7 illustrates a top view and a bottom view of a blood sampling cartridge, according to an example implementation; and

FIG. 8 illustrates another example of a needle, according to an example implementation;

FIG. 9 illustrates one end of a blood sampling cartridge launcher, according to an example implementation.

FIG. 10 illustrates a configuration of a blood sampling cartridge inside a blood sampling cartridge launcher in a hinged position, according to an example implementation;

FIG. 11 illustrates a configuration of a blood sampling cartridge inside a blood sampling cartridge launcher in a hinged position, according to an example implementation;

FIG. 12 illustrates a configuration of a blood sampling cartridge inside a blood sampling cartridge launcher in a hinged position and a blood sample analyzer for connecting to a test strip of the blood sampling cartridge, according to an example implementation;

FIG. 13 illustrates a configuration of a blood sampling cartridge and a gun-type blood sampling cartridge launcher, according to an example implementation;

FIG. 14 illustrates a flowchart depicting a method for operating a blood sampling cartridge, a launcher, and a blood sample analyzer external to the launcher, according to an example implementation; and

FIG. 15 illustrates a flowchart depicting a method for operating a blood sampling cartridge, a launcher, and a blood sample analyzer integrated into the launcher, according to an example implementation.

### DETAILED DESCRIPTION

Implementations of the invention will now be described with reference to the accompanying figures. The terminology used in the description presented herein is not intended to be interpreted in any limited or restrictive manner, simply because it is being utilized in conjunction with a detailed description of certain specific implementations of the invention. Furthermore, implementations of the invention may comprise several novel features, no single one of which is solely responsible for its desirable attributes or which is essential to practicing the inventions herein described.

I. Introduction

### Glucose Measurement

Diabetes patients need to monitor the glucose level in their blood stream. One method is continuous glucose monitoring using a glucose electrode inserted into the body. Another method is a finger prick blood test.

### Finger Prick Blood Test

The finger prick blood test involves a user pricking a finger with a piercing device and contacting the drop of blood formed on the skin with a test strip. After contacting the drop of blood that has formed on the user's skin with the test strip, the user inserts the test strip into a glucose analyzer. In response, the glucose analyzer provides a reading that indicates the user's blood glucose level. For a type-1 diabetes patient, such a finger prick blood test is recommended 4-10 times a day.

### Finger Pricking is Painful

The finger prick method tears a relatively big opening in the patient's finger to provide a sufficient amount of blood for testing. This causes a lot of pain to the patient, multiple times a day. For some patients, the amount of pain can be too much to bear.

### Finger Pricking is Cumbersome

The finger prick blood test involves multiple steps: (1) getting ready all the required tools including the piercing device, test strip, and glucose analyzer; (2) gripping the piercing device and piercing the skin; (3) setting down the piercing device; (4) bringing the test strip in contact with the drop of blood that has formed at the pierced site on the skin; and (5) inserting the test strip into the glucose analyzer. In some cases, the patient may insert the test strip into the glucose analyzer before bringing the test strip in contact with the blood. However, the process of inserting the test strip into the glucose analyzer and contacting the blood with the test strip still requires two separate steps. Performing these multiple steps may be cumbersome. For some older patients, the process may not be easy to perform.

II. Overview

### Blood Sampling Cartridge

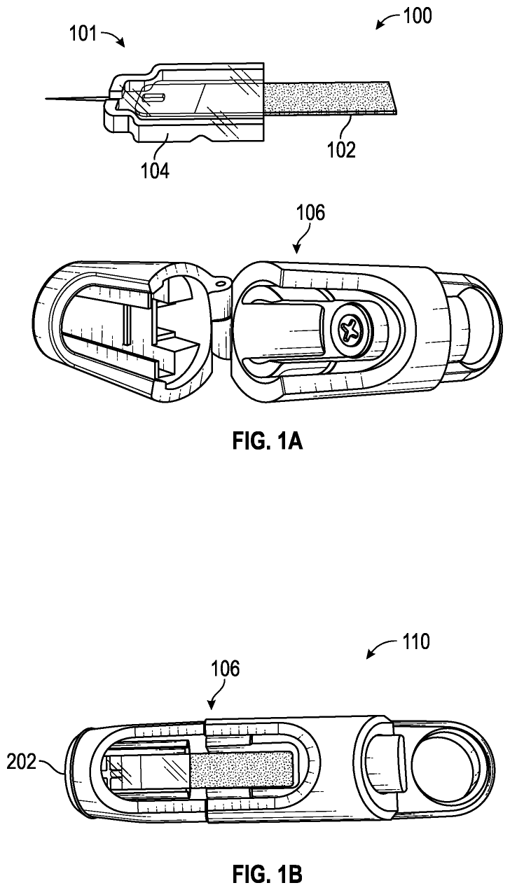

The present disclosure provides a blood sampling cartridge that can be used to collect blood from underneath a user's skin for immediate or in situ blood testing. FIG. 1A illustrates a configuration 100 including a blood sampling cartridge 101 (also referred to herein as the “cartridge”) according to one example implementation. The cartridge 101 includes a blood sampler 104 (also referred to herein as a sampler) and a test strip 102 that has been inserted into the blood sampler 104. As shown in FIG. 1A, the blood sampler 104 includes a needle for piercing the user's skin and a sampler body having an interior space for accommodating the test strip 102 therein.

### Cartridge Launcher

The configuration 100 of FIG. 1A also includes a blood sampling cartridge launcher 106 (also referred to herein as the “launcher”) according to one example implementation. The launcher 106 is designed to be loaded with a blood sampling cartridge such as the cartridge 101 and launch the loaded blood sampling cartridge towards the user's skin to sample blood.

### Cartridge Launcher in Loaded State

FIG. 1B illustrates a configuration 200 in which the cartridge 101 has been loaded in the launcher. In the configuration 200, the needle of the cartridge 101 is hidden inside the launcher 106 and is not be visible to the user. In the example of FIG. 1B, the launcher 106 would launch the cartridge 101 towards the left such that the needle protrudes through a needle hole (not shown) provided on that left end of the launcher 106 for piercing the user's skin.

### Operating Launcher to Launch Cartridge

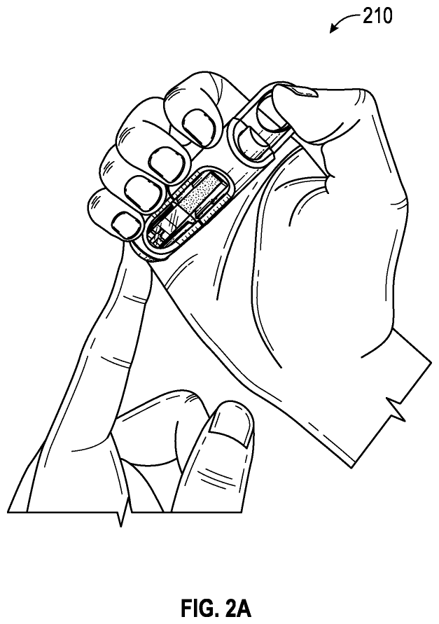

A user might use the cartridge 101 and the launcher 106 for blood sampling and testing as follows: the user loads one cartridge 101 into the launcher 106 to arrive at the configuration 200 illustrated in FIG. 1B; then, the user holds the launcher 106 in the configuration 200 against a target site from which blood will be drawn, such as her fingertip, such that the tip (e.g., a skin-contacting tip 202 shown in FIG. 1B) of the launcher 106 touches the skin at the target site (this is shown in FIG. 2A); and subsequently, the user actuates the launcher 106 to cause the cartridge 101 to be launched towards the skin at the target site (this is shown in FIG. 2B).

### Blood Withdrawal

After the cartridge 101 has been launched, the needle on the cartridge 101 pierces the skin and extracts blood from underneath her skin. The blood travels through the needle to an interior space provided inside the blood sampler 104 in which the test strip 102 is located. The extracted blood then comes in contact with the test strip 102. While this withdrawal of blood is taking place, the user may keep the skin-contacting tip 202 in contact with the subject's skin (e.g., for 1 second, 2 seconds, or any other time period between 1-10 seconds). The user and the subject may be the same person or entity. Alternatively, the user and the subject may be different persons or entities.

### Blood Testing

The test strip 102 can subsequently be analyzed by a glucose analyzer to provide the user with a reading of the user's blood glucose level. In some implementations, the cartridge 101 and launcher 106 are used to test for a material other than glucose.

### Illustration of Cartridge and Launcher Usage

As discussed above, FIGS. 2B and 2C illustrate how a user might hold the launcher 106 when using the cartridge 101 and the launcher 106 to sample her blood, for example, to check her blood glucose levels. FIG. 2A shows the user gripping the launcher 106 and holding the needle end of the launcher 106 against her finger. FIG. 2B shows the user having pressed a launch button provided on the launcher 106 to launch the cartridge 101 towards her finger, which causes the needle provided on the cartridge 101 to pierce the skin on the user's finger. As a result, blood is withdrawn from underneath the user's skin through the needle into the blood sampler 104 (and onto the test strip 101).

III. Blood Sampling Cartridge

### Components of Blood Sampling Cartridge

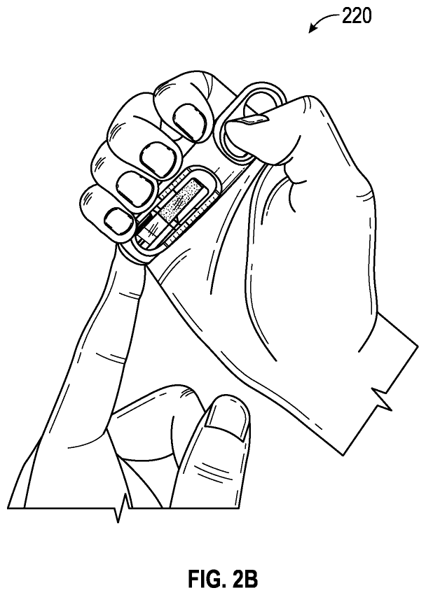

Now that some features of the general operation of the cartridge 101 and launcher 106 have been described, the individual components of the cartridge 101 are described in greater detail below with reference to FIGS. 3A-7. Such components include the test strip 102 and the sampler body 104.

III.a. Test Strip

### Construction of Test Strip

One component of the cartridge 101 is the test strip 102. As illustrated in FIG. 3A according to one example implementation, the test strip 102 has an elongated body that includes a blood contact portion 102A at one end, an analyzer connection portion 102B at the other end, and an intermediate portion 102C located between the two portions.

### Blood Contact Portion

In implementations, the blood contact portion 102A of the test strip 102 includes one or more electrodes specifically designed for detecting a chemical entity contained in blood. For determining glucose levels, the blood contact portion 102A includes a glucose-sensing electrode (not shown) for oxidation-reduction (redox) reaction with glucose molecules. In some implementations, the glucose sensing electrode includes a glucose-specific enzyme. In other implementations, no enzyme is included in the glucose-sensing electrode. The blood contact portion 102A may include one or more additional electrodes for electrochemical reactions for detecting glucose levels. For chemical entities other than glucose, the blood contacting portion 102A includes an electrode for redox reactions with such other chemical entities. One of ordinary skill in the art would understand specific configurations and materials of the electrodes for glucose and other chemical entities.

### Analyzer Connection Portion

In implementations, the analyzer connection portion 102B of the test strip 102 includes contact terminals (not shown) to electrically contact counterpart terminals of a blood sample analyzer. In one example implementation, each contact terminal of the analyzer connection portion 102B corresponds to one of the multiple electrodes formed in the blood contact portion 102A. The analyzer connection portion 102B may be specially designed to connect to only specific blood sample analyzer models or specific adaptors. Alternatively, the analyzer connection portion 102B may be connectable to generic and/or universal analyzer models of one or more types. The analyzer connection portion 102B is also referred to herein as an electric contact terminal portion.

### Intermediate Portion

In implementations, the intermediate portion 102C of the test strip 102 includes wiring that electrically connects the electrodes in the blood contact portion 102A to the corresponding contact terminals in the analyzer connection portion 102B.

### Commercially Available Test Strips

In some implementations, the test strip 102 is a commercially available off-the-shelf test strip. For such off-the-shelf test strips, the sampler body 104B is designed and sized such that when the blood contact portion 102A of the test strip 102 is inserted into the interior space of the blood sampler 104, the blood contact portion 102A can reach an area within the interior space where the blood extracted through the needle of the blood sampler 104 is collected. In other implementations, the test strip 102 is specially designed to fit the blood sampler 104 and to be compatible with the blood sampler 104.

### Alternative Shapes of Test Strip

Although the test strip 102 is illustrated in FIG. 3A as having a straight, elongated body, in other implementations, the test strip can have another shape. One of ordinary skill in the art would understand other variations in the design of the test strip. One of ordinary skill in the art would also understand that the locations of the blood contact portion and/or the analyzer connection portion may vary depending on the design of the test strip.

III.b. Blood Sampler

### Blood Sampler

Another component of the cartridge 101 is the blood sampler 104. As illustrated in FIG. 3A according to one example implementation, the blood sampler 104 includes a sampler body 104B and a needle 104A fixed to the sampler body.

III.b.(i) Needle

### Needle

The needle 104A is to pierce skin and withdraw blood from underneath the subject's skin. The needle has an elongated body extending between a distal end portion for piercing the subject's skin and a proximal end portion attached to the sampler body. FIG. 3B illustrates a close-up 310 of the distal end portion of an example of the needle 104A. As illustrated, the distal end portion may be cut to provide a sharp tip that gradually gets thinner.

### Blood-Flowing Channel

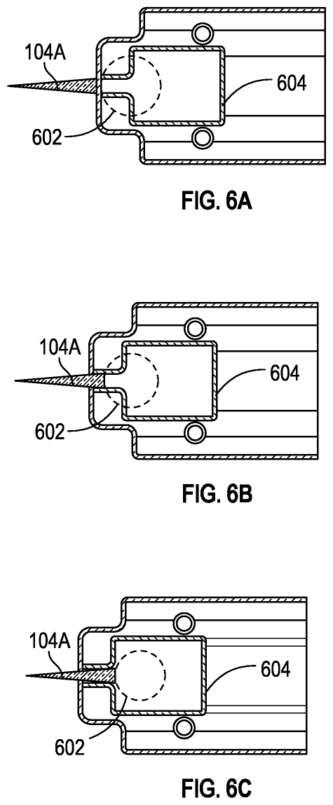

The needle 104A provides a blood-flowing channel extending between the distal end portion and the proximal end portion. As illustrated, the needle 104A has a generally cylindrical shape having a hollow channel inside (as shown in FIG. 3B) for flowing blood from the distal end portion to the proximal end portion (not illustrated in FIG. 3B). In another implementation, the needle 104A of FIG. 8 has a half-cut construction as if the cylindrical body of the needle 310 of FIG. 3B is cut in half along its longitudinal direction. The half-cut construction provides a blood-flowing channel that is open along the longitudinal direction. The term “half-cut construction” refers to any needle constructions that have a blood-flowing channel that is open along the longitudinal direction even if it is not in fact “cut” from a hollow, cylindrical shape and also if it does not have exact one half of a cross-section of a hollow, cylindrical shape.

### Hydrophilic Channel

In implementations, the blood-flowing channel provides a hydrophilic surface for capillary action to cause the blood withdrawn through the needle to flow toward the proximal end of the blood-flowing channel.

### Material and Making of Needle

The needle 104 can be made of a variety of metallic materials, which is well known in the art. A hollow, cylindrical needle is typically manufactured by rolling a metallic sheet. Alternatively, a needle can be made by of polymeric materials injection molding. Bio-compatible polymers having structural strength can be used to produce a needle. To provide more structural strength, one or more coatings can be added on surfaces of a polymeric needle.

### Shape of Needle

As described above, the needle 104A can be a hollow tube. An example of a hollow tube needle is shown in FIG. 3B. In other implementations, the needle 104A is a half-cut tube. An example of a half-cut tube needle is shown in FIG. 8. In other implementations, the cross-section of the needle 104A is a shape other than a full circle (hollow tube) or a half circle (half-cut tube) such as a three-quarters of a circle, a quarter of a circle, an ellipse, to name a few.

### Thickness of Needle

Generally, reducing the size of the outer diameter of the needle reduces the likelihood that the user will experience pain. To reduce or minimize pain, the blood sampler 104 includes a needle that is 30-gauge or above (e.g., inner diameter less than or equal to 150 micrometers, outer diameter less than equal to 300 micrometers), in one example implementation. Such a needle has an increased chance of avoiding or reducing contact with pain spots underneath the user's skin while having a large enough inner diameter to withdraw blood from underneath the user's skin to the blood collection space in the sampler body 104B.

### Length of Needle

The length of the needle 104A is sized such that, when the blood sampler 104 is launched towards the user's skin, the needle 104A penetrates the user's skin deeply enough to extract blood from underneath the user's skin. In some implementations, the length of the needle 104A may be sized such that the penetration depth is in the range of 2 mm to 5 mm, in one example implementation.

Materials of Needle Different from Sampler Body

The needle 104A may be made of a different material than that used for the sampler body 104B. In some implementations, the needle 104 is made of stainless steel. In other implementations, the needle 104A is made of glass or silicon dioxide. In yet other implementations, the needle 104A is made of plastic or other polymers. One of ordinary skill in the art would understand other types of materials that can also be used for the needle 104A.

### Materials of Needle Same as Sampler Body

The needle 104A may be made of the same material(s) used for the sampler body 104B. In some implementations, the needle 104A and the sampler body 104B are both made of stainless steel. In other implementations, the needle 104A and the sampler body 104B are both made of glass or silicon dioxide. In yet other implementations, the needle 104A and the sampler body 104B are both made of plastic or other polymers. One of ordinary skill in the art would understand other types of materials that can also be used for the needle 104A and the sampler body 104B.

### Inner Surface of Needle is Hydrophobic

In some implementations, the inner surface of the needle 104A is hydrophobic, in one example implementation. For example, the inner surface may have a hydrophobicity value that is greater than a threshold value. In such implementations, the inner surface of the needle 104A may be applied a hydrophilic coating, causing the coated inner surface to be hydrophilic (or to have a hydrophobicity value less than the threshold value). For example, the inner surface may be coated using the TiO₂ coating technology of Positive Coatings, a Swiss company. As another example, the inner surface may be coated using the Dursox® method of SilcoTek® Corporation, which involves creating, via chemical vapor deposition (CVD), a chemically protective hydrophilic barrier of amorphous silicon and oxygen. However, in other cases, the hydrophilic coating may be applied using other hydrophilic coating methods, as would be understood by one of ordinary skill in the art. In yet other implementations, a hydrophilic coating is not provided to the inner surface of the needle 104A.

### Inner Surface of Needle is Hydrophilic

In some implementations, the inner surface of the needle 104A is hydrophilic, in one example implementation. For example, the inner surface may have a hydrophobicity value that is less than a threshold value. In some of such implementations, a hydrophilic coating described in the preceding paragraph is not provided to the inner surface of the needle 104A. The hydrophilic nature of the inner surface causes blood at the distal end of the needle 104A to be pulled up through the needle 104A and into the blood collection space of the sampler body 104B by capillary action. In some other implementations, a hydrophilic coating described in the preceding paragraph is applied to the inner surface of the needle 104A despite the inner surface being hydrophilic (e.g., to reduce the hydrophobicity value).

III.b.(ii) Sampler Body

### Sampler Body

Another component of the sampler 104 is the sampler body 104B. The sampler body 104B can be made of plastic, although in other implementations, the sampler body 104B can be made of other materials. As described herein, the sampler body 104B includes an interior space (also referred to herein as inner space) for accommodating the test strip 102. As shown in FIG. 3A, the proximal end portion of the needle 104A is attached to the sampler body 104B.

### Interior Space of Sampler Body

The interior space of the sampler body 104B is formed and defined by inner surfaces of the sampler body 104B. The interior space is in fluid communication with the blood-flowing channel of the needle 104A. Also, the interior space accommodates a leading portion of the test strip 102, including the blood contact portion 102A.

### Blood Collection Area

In implementations, the interior space is designed to allow the blood that is flowing into the interior space to immediately contact the blood contact portion 102A of the test strip 102 without having to flow through additional channels. The interior space includes a blood collection area in which the blood contact portion 102A of the test strip 102B is located when the test strip 102 is fully inserted or engaged with the sampler body 104B. The blood collection area is located at or next to the proximal end of the needle 104A into which the blood withdrawn through the needle 104A flows as the blood comes out of the proximal end of the blood-flowing channel. There may be a gap or distance between the proximal end of the blood-flowing channel and the blood collection area but without a structure that would block or impede the flow of the blood from the proximal end to the blood collection area. Accordingly, when the test strip is fully inserted into the sampler body 104B, the blood withdrawn through the needle 104A reaches the blood contact portion 102A of the test strip 102 as the blood exits the proximal end of the blood-flowing channel of the needle 104A (e.g., without having to flow through another channel).

### Guide for Sliding Test Strip

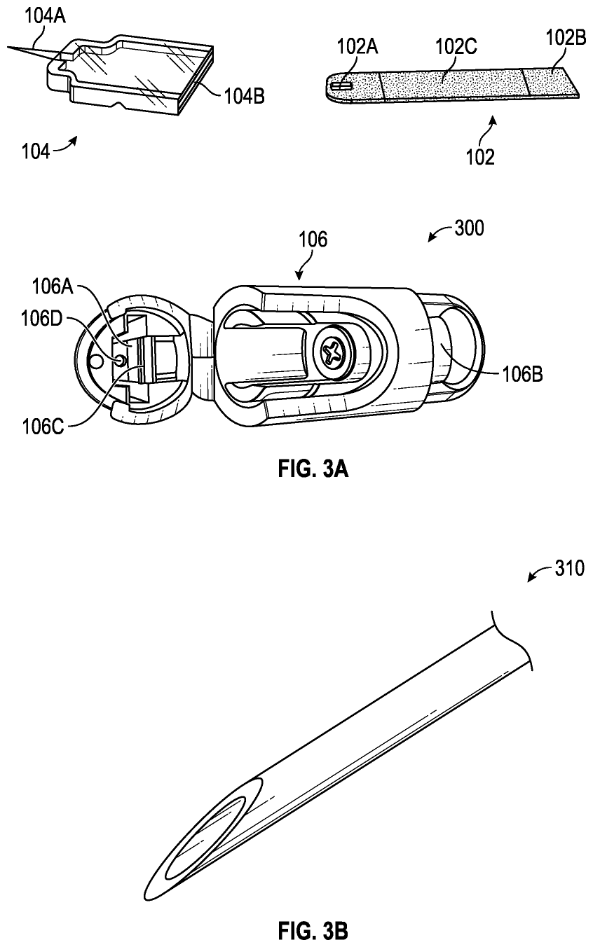

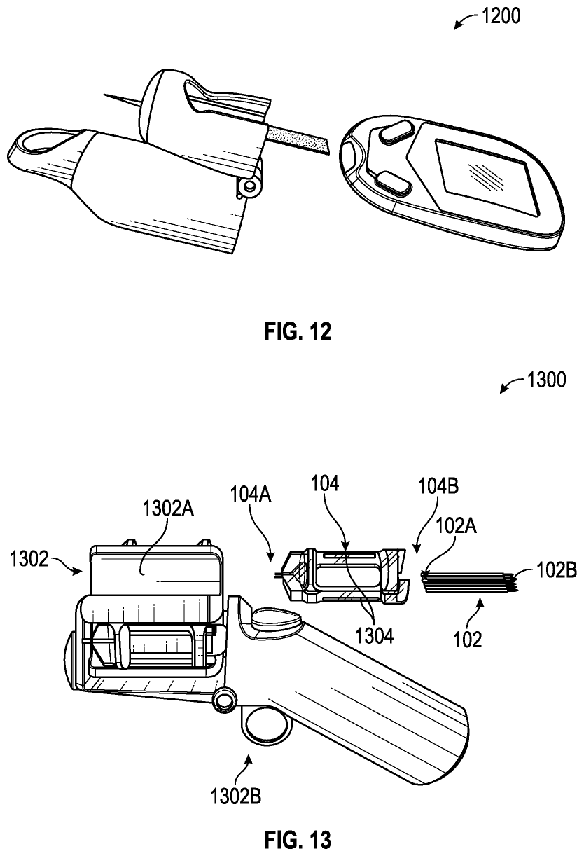

In implementations, the sampler body 104B includes a guide structure to guide sliding of the test strip 102 into the sampler body 104B. The guide is elongated generally along an axis along which the needle 104A attached to the sampler body 104B extends. Accordingly, the elongated body of the test strip 102 is to slide into the sampler body 104B so that the blood contact portion 102A of the test strip 104B can easily reach the blood collection area of the interior space. The guide structure may include the side walls and/or the top/bottom walls that make up the interior space (e.g., top-facing surface 408 and side walls 409 in FIG. 4A) or provided around or near the interior space (e.g., guide members 1304 in FIG. 13). In some implementations, the side walls and/or the top/bottom walls that make up the guide structure may include one or more opening or hollow portions (e.g., as shown in FIG. 13, where the top- and bottom-facing surfaces of the sampler body 104B have large holes that span most of the top- and bottom-facing surfaces).

### Construction of Sampler Body

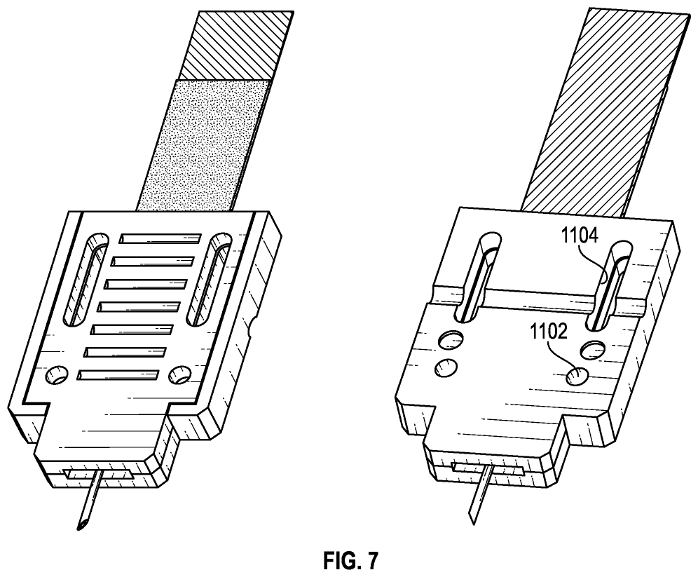

In one example implementation, the needle 104A and the sampler body 104B are made and assembled as shown in FIG. 4A. In the example of FIG. 4A, the sampler body 104B is assembled by combining two pieces of plastic: a top piece 402 and a bottom piece 404. In the example of FIG. 9, the bottom piece 404 includes a recess defined by a top-facing surface 408 and side walls 409 generally perpendicular to the top-facing surface 408. In other implementations, the recess may be provided in one or both of the top piece 402 and the bottom piece 404 to provide the interior space of the sampler body 104B when the pieces are assembled. FIG. 4A also shows a needle attachment piece 406 used to attach the needle 104A to the sampler body 104B. The needle attachment piece 406 is described in greater detail below.

### Inner Surfaces of Interior Space Operate as Test Strip Guide

When the sampler body 104B is assembled, the interior space thereof is defined by the bottom-facing surface (not shown in FIG. 4A) of the top piece 402, the top-facing surface 408 of the bottom piece 404, and the side walls 409 of the bottom piece 404. One or more of these four surfaces alone or collectively (i) guide the test strip 102 as the test strip 102 is inserted into the interior space of the sampler body 104B and (ii) prevent the test strip 102 from turning, rotating, or otherwise moving away from the axis of the needle 104A, thereby operating as a test strip guide.

III.b.(iii) Relationship between Needle and Sampler Body

### Attaching Needle to Sampler Body

As shown in FIG. 4A, the needle 104A can be first attached to an intermediary piece (the needle attachment piece 406 of FIG. 4A), and the intermediary piece can then be combined with the sampler body 104B. Alternatively, the needle 104A can be attached to the sampler body 104B without such an intermediary piece. The needle attachment piece 406 may have a shape corresponding to a groove or space provided in one or both of the upper piece 402 and the bottom piece 404. A close-up of the needle 104A and the needle attachment piece 402 is shown in FIG. 4B.

### Shape of Sampler Body

In the example of FIG. 4A, the portion of the sampler body 104B that would accommodate the needle 104A (e.g., the portion occupied by the needle attachment piece 406) is narrower than the portion of the sampler body 104B that would accommodate the test strip 102 (e.g., the remaining portion of the sampler body 104B not occupied by the needle attachment piece 406). In other implementations, the latter portion may be narrower or those portions may have the same or similar widths.

Needle Made Integrally with Sampler Body

In some implementations, the needle 104A and the sampler body 104B are made integrally using the same process (e.g., as opposed to being made separately and combined). For example, the needle 104A and the sampler body 104B are made by making a top piece and a bottom piece, where the top piece makes up the top half of the needle 104A and the top half of the sampler body 104B, and the bottom piece makes up the bottom half of the needle 104A and the bottom half of the sampler body 104B. The two pieces can be attached to each other to form the needle 104A and the sampler body 104B.

Position of Needle's Proximal End within Sampler Body

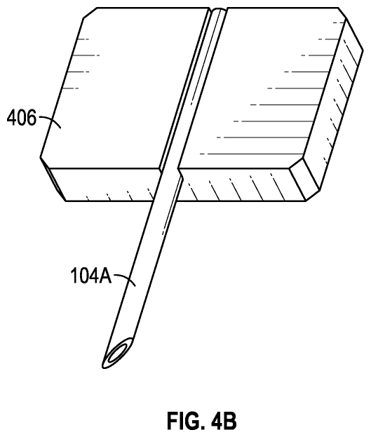

FIGS. 6A-6C illustrate example positions of the needle 104A relative to the sampler body 104B including the interior space. In FIG. 6A, the proximal end (e.g., the end that is closer to the interior space of the sampler body 104B) of the needle 104A is aligned with the end of the sampler body 104B (e.g., the end opposite to that having the opening for receiving the test strip 102), and a blood collection area 602 includes the channel between the proximal end of the needle 104A and the interior space of the sampler body 104B and also a portion of the interior space of the sampler body 104B. In FIG. 6B, the proximal end of the needle 104A is placed between the end of the sampler body 104B and the test strip 102, and the blood collection area 602 includes a portion of the channel between the proximal end of the needle 104A and the interior space of the sampler body 104B and also a portion of the interior space of the sampler body 104B. In FIG. 6C, the proximal end of the needle 104A is in contact with (or almost in contact with) the test strip 102, and the blood collection area 602 includes a portion of the interior space of the sampler body 104B. Although not shown, the proximal end of the needle 104A may be placed inside the interior space such that the needle 104A overlaps with the test strip 102, for example, when viewed from the direction from which the examples of FIGS. 6A-6C are shown. In such a case, the blood collection area includes a portion of the interior space of the sampler body 104B at or near the proximal end of the needle 104A.

Blood Collection Space within Sampler

The space between the needle 104A and the interior space of the sampler body 104B of FIG. 6A may be referred to herein as the blood collection space. In some implementations, the blood collection space may include a portion of the interior space into which blood withdrawn from the user flows.

Needle Elongation Aligned with Blood Collection Space

As shown in FIGS. 6A-6C, the needle 104A is positioned such that the proximal end of the needle 104A points to the blood collection space.

### Pressure Holes on Sampler Body

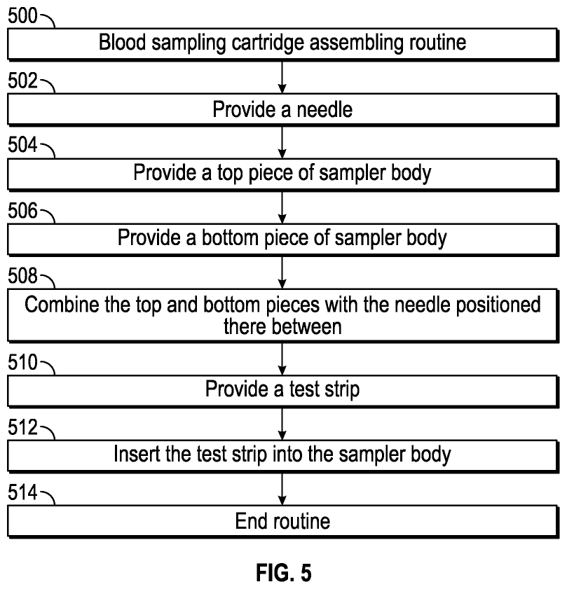

In some implementations, to facilitate the extraction of the user's blood through the needle 104A, the sampler body 104B may include one or more holes that can equalize or reduce the pressure in the interior space of the sampler body 104B. FIG. 7 shows two sets of pressure holes (e.g., circular holes 1102 and elongated holes 1104) on top and bottom views of the cartridge 101.

III.b.(iv) Assembly and Characteristics of Cartridge

### Flowchart for Assembling Blood Sampler Cartridge

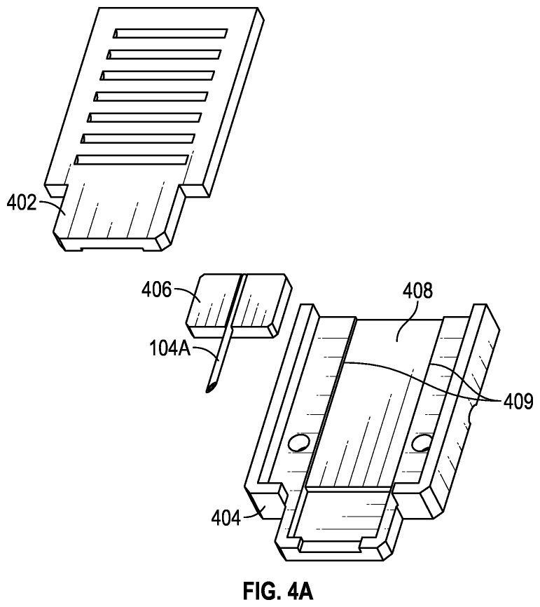

FIG. 5 is a flowchart for assembling the cartridge 101, according to an implementation of the present disclosure. One or more of the steps illustrated in FIG. 5 may be performed by a user manufacturing, supplying, or selling the cartridge 101, by a machine performing the assembly, and/or by an end user using the cartridge 101 (e.g., patient). For convenience, the method 500 is described as performed by a user. The method 500 can include fewer, more, or different blocks than those illustrated in FIG. 5 and/or one or more blocks illustrated in FIG. 5 may be modified, omitted, or switched without departing from the spirit and scope of the description. For example blocks 502-508 may be combined (e.g., by providing a sampler 104).

### Provide Needle

At block 502, the user provides the needle 104A.

### Provide Top Piece of Sampler Body

At block 504, the user provides the top piece 402 of the sampler body 104B.

### Provide Bottom Piece of Sampler Body

At block 506, the user provides the bottom piece 404 of the sampler body 104B.

Combine Top and Bottom Pieces with Needle

At block 508, the user combines the top piece 402 with the bottom piece 404 with the needle 104A positioned therebetween.

### Provide Test Strip

At block 510, the user provides the test strip 102.

### Insert Test Strip

At block 512, the user inserts the test strip 102 into the interior space of the sampler body 104B. At block 514, the method 500 may end.

### Insertion and Usage of Test Strip

When the test strip 102 is inserted into the blood sampler 104 (thereby forming the cartridge 101), the blood contact portion 102A is located inside the interior space of the blood sampler 104 for contacting blood when blood is withdrawn through the needle of the blood sampler 104. The analyzer connection portion 102B, in one example implementation, can be inserted into a blood sample analyzer to analyze the blood collected on the blood contact portion 102A.

### Person(s) Assembling Cartridge

In some implementations, the cartridge 101 is assembled (i.e. test strip 102 is inserted into the blood sampler 104) by the manufacturer (or supplier or seller) prior to being sold to the end user (e.g., patient or clinician). In other implementations, the cartridge 101 is assembled by the end user, by inserting the blood contact portion 102A of the test strip 102 into the interior space in the sampler body 104B, prior to using the cartridge 101. In yet other implementations, the sampler 104 is assembled by the manufacturer (or supplier or seller) and the end user inserts the test strip 102 into the sampler 104 to complete the cartridge 101. For example, the end user may be provided with the sampler 104 and the test strip 102 as a package, and the end user may assemble the cartridge 101. Alternatively, the end user may obtain the sampler 104 and the test strip 102 from separate sources and assemble the cartridge 101.

### Shape and Size of Blood Contact Portion

In the cartridge 101 of FIG. 1A, the blood contact portion 102A is fully inserted into the blood sampler 104. Accordingly, the blood contact portion 102A has a shape and size that allows the blood contact portion 102A to enter the opening provided on the sampler body 104B (e.g., on the right side end of the sampler body 104B in FIG. 3A) and fit inside the interior space of the blood sampler 104.

### Full Insertion of Test Strip: Stopper

In some implementations, to fully insert the test strip 102 into the interior space of the sampler body 104B, the user inserts the tip of the test strip 102 at which the blood contact portion 102A is provided, and pushes the test strip 102 deeper into the interior space of the sampler body 104B until the test strip 102 has traveled a threshold depth into the interior space. For example, the threshold depth is when the tip of the test strip 102 has reached the deepest end of the interior space (e.g., where blood collection area is provided). In this example, the deepest end of the interior space works as a stopper designed to stop the test strip 102 from further traveling deeper into the interior space when the blood contact portion 102A of the test strip 102 has reached the blood collection area of the interior space.

### Full Insertion of Test Strip: Locking Mechanism

As another example, the threshold depth is when a protrusion or locking mechanism provided on the test strip 102 (e.g., the line between the blood contact portion 102A and the intermediate portion 102C in FIG. 3A) has reached and/or surpassed the counterpart recess or locking mechanism provided inside the interior space of the sampler body 104B (e.g., a step 604 shown in FIGS. 6A-6C). For example, the roughly square portion inside the interior space of the sampler body 104B between the step 604 and the blood collection area 602 is designed to accommodate and lock in the corresponding roughly square portion of the test strip 102 on which the blood contact portion 102A is provided). In this example, once the locking mechanism of the test strip has crossed the locking mechanism of the interior space, the test strip 102 has been fully inserted into the interior space and locked into a position where the blood contact portion 102A is in the blood collection area and/or has reached the proximal end of the needle 104A. Although the square shape was used for the locking mechanism, another shape may be used to provide the same locking functionality. The step 604 prevents the test strip 102 from backing out of the interior space once it has engaged with the counterpart locking mechanism provided on the test strip 102.

Blood Contact Portion of Test Strip Reaches Blood Collection Space when Inserted

In some implementations, when the test strip 102 has been fully inserted into the sampler body 104B, the proximal end of the needle 104A is in contact with the blood contact portion 102A of the test strip 102 such that the blood at the proximal end of the needle 104A reaches the blood contact portion 102A of the test strip 102 by capillary action. In other implementations, when the test strip 102 has been fully inserted into the sampler body 104B, the proximal end of the needle 104A is not in contact with the blood contact portion 102A of the test strip 102 but close enough to the blood contact portion 102A of the test strip 102 such that the blood at the proximal end of the needle 104A reaches the blood contact portion 102A of the test strip 102 by capillary action.

### Insertion of Test Strip via Guide

Once slid into the sampler body 104B guided by the guide structure provided in the sampler body 104B, the tip of the blood contact portion 102A reaches the proximal end of the needle 104A or reach the blood collection area that is in fluid communication with the proximal end of the needle 104A. This design allows the blood extracted from underneath the user's skin through the needle 104A to reach the blood contact portion 102A (directly or via the inner space provided therebetween), and thereby eliminates the need to manually bring the test strip 102 in contact with the drop of blood that has formed on the user's skin, as done in the finger prick method described above.

Needle Elongation Aligned with Test Strip

As shown in FIGS. 6A-6C, the interior space is designed such that the blood contact portion 102A of the test strip 102, when inserted into the interior space, points to and reaches the blood collection space inside the sampler body 104B.

### Analyzer Connection Portion Exposed Outside Interior Space of Sampler

As shown in FIG. 1A, the interior space of the sampler body 104B can be deep enough to cover about half of the test strip 102. For example, when the test strip 102 has been fully inserted into the interior space, the blood contact portion 102A may be inside the sampler body 104B (e.g., in the interior space provided therein) while the analyzer connection portion 102B is outside the sampler body 104B. Thus, event after the blood contact portion 102A has been inserted into the sampler body 104B, the analyzer connection portion 102B remains outside the sampler body 104B. Alternatively, in other implementations, when the test strip 102 has been fully inserted into the sampler body 104B, both of the blood contact portion 102A and the analyzer connection portion 102B are inside the sampler body 104B.

### Exposed Analyzer Connection Portion Allows Convenient Sampling and Testing

The depth of the interior space of the sampler body 104B allows a blood sample analyzer to be connected to the analyzer connection portion 102B while the blood contact portion 102A is still inside the sampler body 104B. This design of the sampler body 104B allows both the blood sampling and the blood testing to occur without requiring the user to move (or otherwise maneuver) the test strip 102. In contrast, in the finger prick method described above, after blood is withdrawn and a drop of blood is formed on the user's skin, the user is required to bring the test strip to the drop of blood to collect blood onto the test strip by touching the drop of blood with the test strip.

### Locking Mechanism Inside Sampler Body

Inside the sampler body 104B, a locking mechanism may be provided for locking the test strip 102 inside the sampler body 104B. Once the test strip 102 is locked into the sampler body 104B by the locking mechanism, removing the test strip 102 from the sampler body 104B requires a much greater amount of force than that used to insert the test strip 102 into the sampler body 104B. The test strip 102 may include a corresponding groove or protrusion that may be caught by the locking mechanism.

### Snug Fit of Test Strip Inside Sampler Body

In some implementations, the width of the blood contact portion 102A does not allow the test strip 102 to be wiggled within the interior space of the sampler body 104B and the axis of the test strip 102 remains aligned with the axis of the sampler body 104B as long as the blood contact portion 102A is inside the sampler body 104B. In some implementations, the height of the blood contact portion 102A is design such that the top surface and the bottom surface are both touching the walls of the interior space of the sampler body 104B (e.g., so that the locking mechanism can be properly engaged).

Removing Test Strip from Blood Sampler

As shown in FIG. 3A, the test strip 102 can be removed from the blood sampler 104. The user may hold the analyzer connection portion 102B of the test strip 102 and pull the test strip 102 away from the blood sampler 104 to remove the test strip 102 from the blood sampler 104. The sampler body 104B of the blood sampler 104 may include a locking mechanism that requires the test strip 102 to be pulled away from the sampler body 104B with a threshold amount of force (e.g., greater than that required to insert the test strip 102 into the sampler body 104B) in order to be removed from the sampler body 104B. After the test strip 102 has been removed from the blood sampler 104, the blood contact portion 102A of the test strip 102 may still have the blood collected via the needle 104A of the blood sampler 104. Alternatively, in other implementations, the test strip 102 is not removable from the blood sampler 104 by pulling on the test strip 102.

### Disposable Cartridge

The cartridge 101 may be designed to be disposed after a single use (e.g., after collecting blood on the test strip and testing the collected blood). In some implementations, the sampler body 104B is designed such that the removal of the test strip 102 from the sampler body 104B damages the blood contact portion 102A of the test strip 102 such that a test strip reader or a blood sample analyzer can no longer take measurements from the blood collected on the blood contact portion 102A (or such that the test strip 102 cannot be used again). For example, the locking mechanism provided inside the interior space may damage the test strip 102 as the test strip 102 is forcibly pulled out of the interior space. In other implementations, the sampler body 104B of the blood sampler 104 is not designed to damage the test strip 102 in such a manner.

IV. Launcher

### Components of Blood Sampling Cartridge Launcher

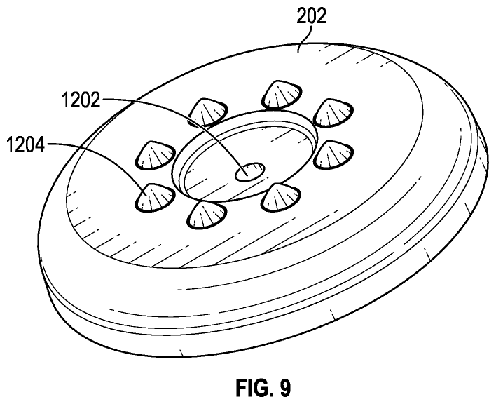

Now that some features of the components of the cartridge 101 have been described, the individual components of the launcher 106 are described in greater detail below with reference to FIGS. 1B, 3A, and 9-15.

### Launcher

Turning back to FIG. 3A, in one example implementation, the launcher 106 includes a cartridge holder 106A, a launching mechanism 106B, a protrusion 106C, and a needle hole 106D. The cartridge holder 106A is used for receiving and holding the cartridge 101. The launching mechanism 106B is used for launching the cartridge 101 towards the user's skin. The protrusion 106C is for positioning the cartridge 101 within the cartridge holder 106A for launching. The needle hole 106D is used for letting the needle 104A through.

### Cartridge Holder

The cartridge holder 106A is used for receiving and holding the cartridge 101. The cartridge holder 106A has an opening into which the sampler body 104B of the cartridge 101 can be inserted.

### Cartridge Guide

The cartridge guide (not labeled) includes one or more guide surfaces that guide the sampler body 104B as the sampler body 104B slides into the opening provided by the cartridge holder 106A.

### Launching Mechanism

The launching mechanism 106B is used for launching the cartridge 101 towards the user's skin.

### Protrusion

The protrusion 106C is for positioning the cartridge 101 within the cartridge holder 106A for launching. In some implementations, the protrusion 106C stops the sampler body 104B before the sampler body 104B reaches the deepest end of the interior space inside the cartridge holder 106A.

### Needle Hole on Skin-Contacting Tip of Launcher

The needle hole 106D (also referred to herein as a through hole) is used for letting the needle 104A of the cartridge 101 through. The width (or inner diameter) of the hole is greater than the width (or outer diameter) of the needle 104A. As another example, FIG. 9 shows a needle hole 1202 provided at the skin-contacting tip 202 of the launcher 106.

### Protrusions on Skin-Contacting Tip of Launcher

FIG. 9 shows protrusions 1204 (e.g., the pointy objects shown around the needle hole 1202 in FIG. 9). Having such protrusions 1204 touch the user's skin prior to the needle 104A entering the user's skin may lessen the pain associated with the needle 104A perceived by the user.

### Hidden Needle Projects Upon Actuation

After loading of the cartridge 101 into the launcher 106 but before launching the cartridge 101, the distal end of the needle 104A does not project through the hole. When the cartridge 101 is launched by the launching mechanism 106B, the distal end of the needle 104A projects through the hole towards and into the user's skin. In some implementations, in response to the user's actuation of the launching mechanism 106B, the distal end of the needle 104A projects about 2 mm to 5 mm outside the hole, in one example implementation.

### Cartridge Loading Space Inside Launcher

The user can insert the cartridge 101 into the cartridge holder 106A, with the needle 104A end first. FIG. 1B illustrates a configuration 110 in which the cartridge 101 has been inserted into the cartridge holder 106A of the launcher 106.

Guide inside Cartridge Loading Space

The cartridge holder 106A includes, in one example implementation, a guide that guides the manner in which the cartridge 101 is inserted into the launcher 106 and also guides the manner in which the cartridge is launched towards the target site. The guide allows the needle 104A to be aligned with the needle hole provided at the end of the launcher 106 when the cartridge 101 has been inserted into the launcher 106, and allows the needle 104A to be launched through the needle hole along the axis of the launcher 106 (direction in which the user has aimed the launcher 106).

### Stopper Inside Cartridge Loading Space

The cartridge holder 106A includes, in one example implementation, a stopper that stops the cartridge 101 after the cartridge 101 has been inserted into the cartridge holder 106A by a threshold or predetermined depth. In some implementations, the threshold depth is one that allows the cartridge 101 to be within the reach of the launching mechanism 106B and also allows some room between the deepest end of the cartridge loading space and the cartridge 101 such that the cartridge 101 can still be launched towards the deepest end of the cartridge loading space (and the needle 104A towards the needle hole) when the user actuates the launching mechanism 106B. The cartridge 101 may include a corresponding groove or protrusion that may be caught by the stopper.

### Example of Stopping Mechanism

One example of such a stopping mechanism is shown in FIG. 3A as the protrusion 106C, which corresponds to a counterpart recess (not labeled) provided on the bottom surface of the sampler body 104 in FIG. 3A. In this example, when the recess provided on the sampler body 104 engages with the protrusion 106C upon insertion of the sampler body 104 into the opening 106A of the launcher 106, the sampler body 104 is stopped by the engagement of the recess and the protrusion 106C from being inserted deeper into the opening 106A (unless a threshold amount of force is applied in the direction into the opening 106A). When the sampler body 104 is stopped, there is remaining space between the deepest end of the opening 106A (e.g., near the needle hole 106D) and the portion of the sampler body 104 that has been inserted deepest into the opening 106A. This remaining space allows the sampler body 104 to be projected even deeper into the opening 106A for allowing the needle 104A to be moved through the needle hole 106D and pierce the subject's skin for withdrawing blood from underneath the subject's skin.

### Launching Mechanism of Launcher

In some implementations, the launching mechanism 106B is manually operated by pressing on the button shown in FIGS. 2A and 3A such that the needle 104A moves as much as the button is moved. In other implementations, the launching mechanism 106B is spring loaded such that when the button is pressed with a sufficient amount of force to be actuated, the needle 104A instantaneously launches into the user's skin with the same amount of force regardless of how much force applied on the button for how long.

### Cartridge Pusher on Launching Mechanism

In some implementations, the launching mechanism 106B includes a cartridge pusher that pushes the cartridge 101 deeper into the cartridge holder 106A. When the cartridge 101 has been inserted into the cartridge holder 106A, the end of the sampler body 104B (e.g., the end opposite to the needle 104A) is in contact with the cartridge pusher. In some implementations, in response to the user actuating the launching mechanism 106B, the cartridge pusher pushes the sampler body 104B without touching the test strip 102 inserted into the cartridge 101. For example, the cartridge pusher may push outer portions of the surface of the sampler body 104B that is facing away from the needle 104A that are not blocked or occupied by the test strip 102.

Removing Cartridge from Launcher

The user may hold the cartridge 101 and pull out the cartridge 101 from the cartridge holder 106A while the test strip 102 is still inserted into the cartridge 101. The cartridge holder 106A may include a locking mechanism that requires the cartridge 101 to be pulled away from the cartridge holder 106A with a threshold amount of force (e.g., greater than that required to insert the cartridge 101 into the cartridge holder 106A) in order to be removed from the cartridge holder 106A. When the cartridge 101 has been removed from the launcher 106, the needle 104A that was used to pierce the user's skin may still be attached to the cartridge 101, and the blood contact portion 102A of the test strip 102 may have the user's blood thereon.

Retraction of Needle Back into Launcher

In some implementations, the launcher 106 is designed such that when the cartridge 101 is launched by the launcher 106, the needle 104A of the cartridge 101 enters the user's skin, withdraws blood, and retracts back to the original position. In other implementations, the launcher 106 is designed such that when the cartridge 101 is launched by the launcher 106, the needle 104A of the cartridge 101 enters the user's skin and stays at least partially under the user's skin until the needle 104A is pulled out manually by the user (e.g., by pulling the launcher 106 away from the user's skin).

### Hinged Launcher

In some implementations, the launcher 106 has a hinge (as shown in FIGS. 1-6). In other implementations, the launcher 106 does not have a hinge (e.g., as shown in FIG. 13).

### Hinged Launcher in Straight/Locked Position

In FIG. 1B, the blood sampling cartridge is in its straight position (may also be referred to as locked position). In the straight position, the cartridge 101 inside the opening of the launcher 106 is locked into the opening of the launcher 106 and is not slidably removable from the blood sampling cartridge 101 because in the straight position, the launching mechanism 106B is blocking the cartridge 101 and is locking the cartridge 101 in the cartridge holder 106A of the launcher 106.

### Cavity for Accommodating Test Strip in Straight Position

While the launcher 106 is in the straight position, the analyzer connection portion 102B of the test strip 102 is accommodated in a cavity created in the launcher 106 such that the analyzer connection portion 102B of the test strip 102 is protected from inadvertent contact by the user.

### Hinged Launcher in Partially Hinged Position

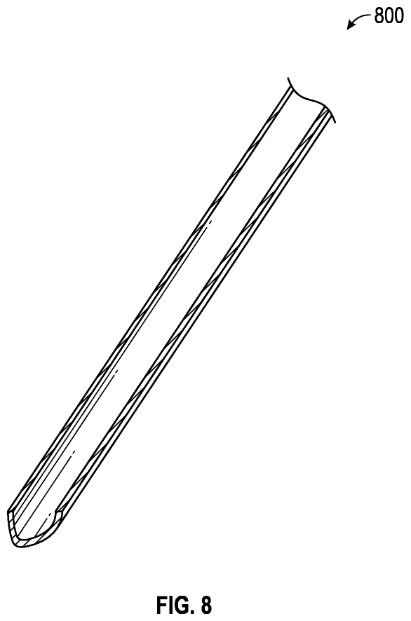

FIG. 10 illustrates the launcher 106 in a partially hinged position in which the launching mechanism is no longer aligned with the opening of the blood sampling cartridge and is no longer blocking the opening of the launcher 106. When the launcher 106 is in the partially hinged position, the blood sampling cartridge 101 can be inserted into and/or removed from the opening of the launcher 106. In the hinged position, the launcher 106 exposes the analyzer connection portion of the test strip 102 of the cartridge 101 so that a blood sample analyzer can be connected thereto.

### Blood Sample Analyzer

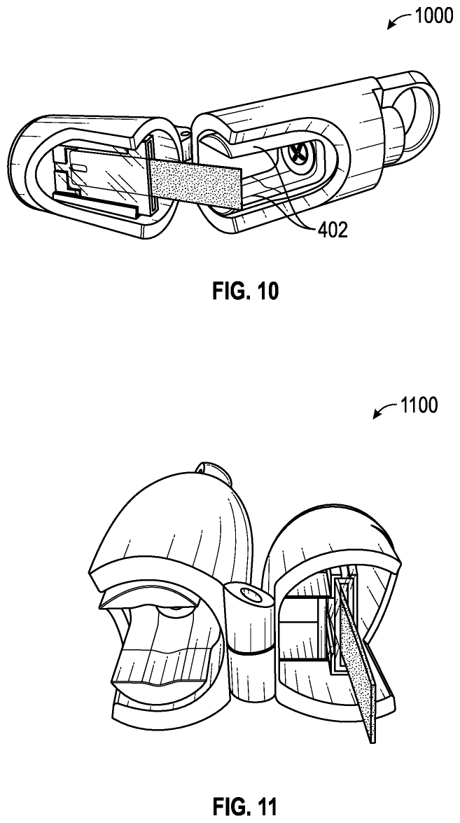

A blood sample analyzer can be connected to the analyzer connection portion 102B of the test strip while the launcher 106 is in the hinged position as shown in FIG. 12. Alternatively, a blood sample analyzer may be integrated into the launcher 106 and may be connected to the analyzer connection portion 102B while the launcher 106 is in the straight position as shown in FIG. 1B.

### Blood Testing Facilitated by Launcher

In addition to launching the cartridge 101 into the user's skin for purposes of collecting the user's blood onto the test strip 102, the launcher 106 facilitates the testing of the blood sample collected onto the test strip 102. For example, as shown in FIGS. 4-6, the launcher 106 may be designed (e.g., designed to include a hinge mechanism) such that a blood sample analyzer can be connected to the analyzer connection portion 102B of the test strip 102 without having to remove the cartridge 101 from the launcher 106 (e.g., by converting the launcher 106 into a hinged position). As another example, the launcher 106 may include an integrated blood sample analyzer that connects to the analyzer connection portion 102B either (i) even before the cartridge 101 is launched to collect blood from the user (e.g., the gun launcher shown in FIG. 13) or (ii) in response to user activation after the cartridge 101 is launched and blood is withdrawn.

### Analyzer External to Launcher

In some implementations, a blood sample analyzer is not connected to the analyzer connection portion 102B of the test strip 102 at the time the needle 104A of the cartridge 101 enters the user's skin. Instead, the user may connect a blood sample analyzer to the analyzer connection portion 102B of the test strip 102 after blood has been collected onto the blood contact portion 102A of the test strip 102. In such implementations, piercing the user's skin and collecting the user's blood onto the test strip 102 may be done in response to the user's actuation of the launch button on the launcher 106, and subsequently, the blood collected onto the test strip 102 may be tests in response to the user performing another action (e.g., connecting the blood sample analyzer to the analyzer connection portion 102B of the test strip 102 and/or actuating a button on the blood sample analyzer).

### Flowchart for Operating Cartridge, Launcher, and External Analyzer

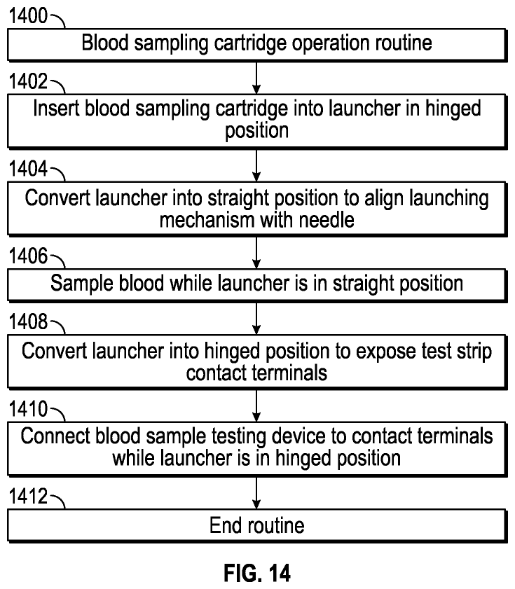

FIG. 14 is a flowchart for operating the cartridge 101 and the launcher 106, according to an implementation of the present disclosure. The steps illustrated in FIG. 14 may be performed by a patient operating the blood sampling cartridge 101 and the launcher 106 to monitor his or her glucose levels. For convenience, the method 800 is described as performed by a user. The method 800 can include fewer, more, or different blocks than those illustrated in FIG. 14 and/or one or more blocks illustrated in FIG. 14 may be modified, omitted, or switched without departing from the spirit and scope of the description.

Inserting Blood Sampling Cartridge into Launcher

At block 802, the user inserts the cartridge 101 into the launcher 106 while the launcher 106 is in the hinged position.

Transform Launcher into Straight Position

At block 804, the user transforms the launcher 106 into the straight position to align the launching mechanism 106B with the needle 104A.

### Sample Blood in Straight Position

At block 806, the user samples blood while the launcher 106 is in the straight position by actuating the launching mechanism 106B.

Transform Launcher into Hinged Position

At block 808, the user transforms the launcher 106 into the hinged position to expose the analyzer connection portion 102B of the test strip 102.

### Connect to Blood Sample Analyzer in Hinged Position

At block 810, the user connects a blood sample analyzer to the analyzer connection portion 102B of the test strip 102 while the launcher 106 is in the hinged position. At block 812, the method 800 may end.

### Display Analyzer Reading

After the sampled blood has been analyzed by the analyzer, a reading (e.g., blood glucose level) may be displayed on a display of the analyzer.

Analyzer Integrated into Launcher

In some implementations, a blood sample analyzer is connected to the analyzer connection portion 102B of the test strip 102 at the time the needle 104A of the cartridge 101 enters the user's skin such that blood collected onto the blood contact portion 102A is immediately tested by the blood sample analyzer without further action by the user. In such implementations, after piercing the user's skin, no additional user action is needed to extract and test the blood. For example, by actuating the launch button on the launcher 106 while the blood sampling cartridge 101 is loaded onto the launcher 106 and while the launcher 106 is aimed at the user's skin, the user may be able to pierce her skin, collect blood onto the test strip, and perform tests on the collected blood.

### Flowchart for Operating Cartridge, Launcher, and Integrated Analyzer

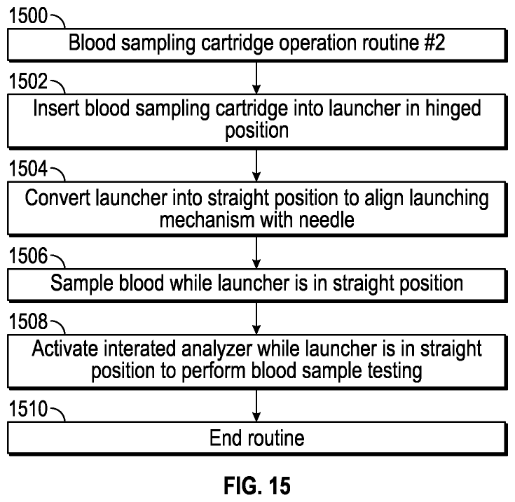

FIG. 15 is a flowchart for operating the cartridge 101, the launcher 106, and a blood sample analyzer integrated into the launcher 106, according to an implementation of the present disclosure. The steps illustrated in FIG. 15 may be performed by a patient operating the cartridge 101, launcher 106, and the integrated analyzer to monitor his or her glucose levels. For convenience, the method 1500 is described as performed by a user. The method 1500 can include fewer, more, or different blocks than those illustrated in FIG. 15 and/or one or more blocks illustrated in FIG. 15 may be modified, omitted, or switched without departing from the spirit and scope of the description.

Inserting Blood Sampling Cartridge into Launcher

At block 1502, the user inserts the cartridge 101 into the launcher 106 while the launcher 106 is in the hinged position.

Transform Launcher into Straight Position

At block 1504, the user transforms the launcher 106 into the straight position to align the launching mechanism 106B with the needle 104A.

### Sample Blood in Straight Position

At block 1506, the user samples blood while the launcher 106 is in the straight position by actuating the launching mechanism 106B.

### Activate Integrated Analyzer

At block 1508, the user activates the integrated analyzer while the launcher 106 is in the straight position to perform blood sample testing on the sampled blood. At block 1510, the method 1500 may end.

### Automatic Activation of Analyzer

In another implementation, the integrated analyzer does not require activation to perform the blood sample testing, and automatically initiates the blood sample once the user's blood has been collected onto the test strip 102.

### Display Analyzer Reading

After the sampled blood has been analyzed by the analyzer, a reading (e.g., blood glucose level) may be displayed on a display of the analyzer.

V. Additional Use Cases, Implementations, and Considerations

### Additional Sampler Use Case: Blood Sample Transport

In some implementations, the sampler 104 is used without a test strip. For example, the user's blood is extracted through the needle 104A and collected inside the interior space of the sampler 104. The sampler 104 including the user's blood is then used to perform testing on the blood sample collected inside the interior space of the sampler 104 using a blood analyzer (e.g., by the same user at the same site), or to transport the user's blood to a different location (e.g., off-site location and/or by a different operator) for subsequent testing at a later time.

### Additional Sampler Use Case: Drug Delivery

In some implementations, the sampler 104 is used to deliver a drug through the needle into the user's body. For example, the interior space of the sampler 104 may not have a test strip 102 inserted therein and instead be filled with a drug or another type of liquid to be delivered to the user's body. After the sampler 104 is inserted into the launcher 106 and launched towards the user's skin, the needle 104A pierces the user's skin and delivers the drug from the interior space of the sampler 104 into the user's body. In such cases, the sampler body 104B may not include an opening (e.g., other than the opening through the needle). Alternatively, the sampler body 104B may include an opening described herein (e.g., opening into which the test strip 102 is described as being inserted).

### Non-Glucose Applications

In addition to blood glucose testing, the blood sampling cartridge 101 and the launcher 106 may be used in connection with other types of blood sampling and testing such as cholesterol testing, nicotine testing, HIV testing, and the like.

### Example Implementations (EIs)

Some enumerated example implementations (EIs) are provided in this section, without limitation.

EI 1: A blood sampling cartridge comprising: a blood test strip extending between a first end and a second end along a longitudinal axis thereof, the blood test strip comprising (i) a blood contact portion at or adjacent the first end and (ii) an electric contact terminal portion at or adjacent the second end; a blood sampler comprising a sampler body and a needle fixed to the sampler body; the needle configured to withdraw blood from underneath a subject's skin and to cause the withdrawn blood to flow to the sampler body and comprising a distal end and a proximal end; and the sampler body comprising a front end and a back end along a longitudinal axis thereof, the sampler body further comprising an inner space and a back opening formed at or toward the back end, wherein the needle is fixed to the sampler body such that the proximal end of the needle is located inside the sampler body and in fluid communication with the inner space, wherein the back opening configured to receive the first end of the blood test strip for inserting the first end of the blood test strip into the inner space, wherein the sampler body further comprises a guide extending along the longitudinal axis of the sampler body and configured to guide traveling of the blood test strip in a first direction of the longitudinal axis from the back end toward the front end, wherein the inner space further comprises a blood collection area located at or adjacent the proximal end of the needle into which blood withdrawn through the needle flows out of the proximal end of the needle, wherein the sampler body comprises a stopper configured to stop the blood test strip from further traveling in the first direction when the blood contact portion of the blood test strip reaches the blood collection area of the inner space, at which the blood test strip is fully inserted into the inner space, wherein the sampler body and the blood test strip are configured such that, when the blood test strip is fully inserted into the inner space, the electric contact terminal portion of the blood test strip remains exposed outside the back opening.

EI 2: The blood sampling cartridge of any preceding EI or any combination of the preceding EIs, wherein the needle comprises a channel with hydrophilic surfaces configured to cause capillary action to cause the blood to flow toward the proximal end.

EI 3: The blood sampling cartridge of any preceding EI or any combination of the preceding EIs, wherein the sampler body comprises at least one through hole for providing fluid communication between the inner space and outside the sampler body such that air pressure within the inner space is released through the at least one through hole as blood withdrawn through the needle flows into the inner space.

EI 4: The blood sampling cartridge of any preceding EI or any combination of the preceding EIs, wherein the sampler body comprises a needle attachment piece to which a portion of the needle is fixed, a first piece and a second piece that are integrated together, wherein the needle attachment piece is received at a predetermined location between the first piece and the second piece, wherein the first piece and the second piece together define the inner space.

EI 5: The blood sampling cartridge of any preceding EI or any combination of the preceding EIs, wherein the sampler body further comprises a mechanism configured to engage with a counterpart mechanism of the blood test strip for inhibiting the blood test strip from being pulled out of the inner space in a second direction opposite to the first direction after the blood test strip has been fully inserted into the inner space.

EI 6: The blood sampling cartridge of any preceding EI or any combination of the preceding EIs, wherein the sampler body further comprises a mechanism configured to damage the blood test strip when the blood test strip from is pulled out of the inner space in a second direction opposite to the first direction after the blood test strip has been fully inserted into the inner space.

EI 7: The blood sampling cartridge of any preceding EI or any combination of the preceding EIs, wherein the blood test strip is fully inserted into the inner space, and the blood contact portion of the blood test strip is at the blood collection area of the inner space such that blood withdrawn through the needle is to immediately contact the blood contact portion of the blood test strip.

EI 8: A method of making the blood sampling cartridge of any preceding EI or any combination of the preceding EIs, the method comprising: providing the blood test strip; providing the blood sampler comprising the sampler body and the needle fixed to the sampler body; inserting the first end of the blood test strip through the back opening of the sampler body; and subsequently continuing to insert the blood test strip into the sampler body by pushing the blood test strip further into the back opening generally in the first direction until the blood test strip is stopped.

EI 9: An apparatus for launching the blood sampling cartridge of any preceding EI or any combination of the preceding EIs, the apparatus comprising: a first body elongated along a first axis and comprising: a skin-contacting tip located at a front end of the first body along the first axis and comprising a through hole, a rear opening located at a rear end of the first body along the first axis and configured to receive the blood sampling cartridge, and a cartridge guide formed inside the first body and configured to guide traveling of the blood sampler of the blood sampling cartridge, subsequent to insertion via the rear opening, in a first direction of the first axis from the rear end toward the front end; and a second body elongated along a second axis and comprising an actuator configured to exert force along the second axis, wherein the first body and the second body are hingedly connected to hingedly move between a straight configuration and a bent configuration, wherein the first axis and the second axis are aligned in the straight configuration, whereas the first axis and the second axis are not aligned in the bent configuration, wherein, in the straight configuration, the second body blocks the rear opening of the first body such that the blood sampling cartridge cannot be inserted into the cartridge guide in the straight configuration, wherein, in the bent configuration, the first axis and the second axis are not aligned, and the second body does not block the rear opening of the first body such that the blood sampling cartridge is insertable into the cartridge guide through the rear opening of the first body in the bent configuration, wherein, upon inserting the blood sampling cartridge into the cartridge guide through the rear opening in the bent configuration, the distal end of the needle stays within the first body, wherein, in the straight configuration with the blood sampling cartridge inserted into the cartridge guide, the actuator of the second body is configured to exert force on the sampler body of the blood sampling cartridge along the second axis to cause the blood sampling cartridge to travel in the first direction along the cartridge guide so that the distal end of the needle advances in the first direction through the through hole of the skin-contacting tip of the first body.

EI 10: The apparatus of any preceding EI or any combination of the preceding EIs, wherein the second body further comprises a blood analyzer comprising a counterpart electric contact terminal portion configured to electrically connect to the electric contact terminal portion of the blood test strip.

EI 11: The apparatus of any preceding EI or any combination of the preceding EIs, wherein the counterpart electric contact terminal portion is configured to slide along the second axis of the second body for engaging with the electric contact terminal portion of the blood test strip when the apparatus is in the straight configuration.

EI 12: The apparatus of any preceding EI or any combination of the preceding EIs, wherein the apparatus in the straight configuration is configured for gripping with one hand, wherein the actuator comprises a push button formed on the second body for pressing with a finger of the hand and a mechanism configured to transfer force from pressing the push button to the sampler body of the blood sampling cartridge.

EI 13: The apparatus of any preceding EI or any combination of the preceding EIs, wherein the actuator comprises a spring-loaded actuator comprising a spring mechanism configured to store spring energy for generating the force for exertion on the sampler body, wherein the second body further comprises a trigger configured to initiate a release of the spring energy.

EI 14: The apparatus of any preceding EI or any combination of the preceding EIs, wherein the spring mechanism is configured to store the spring energy in response to the first body and the second body hingedly moving from the bent configuration to the straight configuration such that the trigger is ready to initiate the release of the spring energy upon turning to the straight configuration.

EI 15: The apparatus of any preceding EI or any combination of the preceding EIs, wherein the blood sampling cartridge is inserted into the cartridge guide of the first body, wherein the electric contact terminal portion of the blood test strip remains exposed outside the back opening of the sampler body of the blood sampling cartridge.

EI 16: A method of sampling blood, the method comprising: providing the blood sampling cartridge of any preceding EI or any combination of the preceding EIs, in which the blood test strip is fully inserted into the inner space; providing the apparatus of any preceding EI or any combination of the preceding EIs; inserting the blood sampling cartridge into the cartridge guide through the rear opening of the first body while the apparatus is in the bent configuration in which the second body does not block the rear opening of the first body, wherein, upon inserting the blood sampling cartridge into the cartridge guide through the rear opening in the bent configuration, the distal end of the needle is positioned between the rear opening and the through hole of the first body; subsequently configuring the apparatus into the straight configuration; holding the apparatus in the straight configuration with one hand; positioning the skin-contacting tip at the front end of the first body against the subject's skin; and causing the actuator to exert force on the sampler body of the blood sampling cartridge to cause the blood sampling cartridge to travel in the first direction and the distal end of the needle to advance through the through hole of the skin-contacting tip for piercing into the subject's skin, such that blood is withdrawn from underneath the subject's skin the blood flows through the needle by capillary action to the blood collection area of the inner space and reaches the blood contact portion of the blood test strip in the inner space.

EI 17: The method of any preceding EI or any combination of the preceding EIs, wherein throughout the steps of the method, the electric contact terminal portion of the blood test strip remains outside the back opening of the sampler body of the blood sampling cartridge.

EI 18: The method of any preceding EI or any combination of the preceding EIs, further comprising: subsequent to causing the actuator to exert force on the sampler body, maintaining the skin-contacting tip in contact with the subject's skin for a period between one second and 10 seconds for completing the withdrawal of the blood.

EI 19: The method of any preceding EI or any combination of the preceding EIs, further comprising: subsequent to causing the actuator to exert force on the sampler body, distancing the apparatus from the subject's skin; subsequently, reconfiguring the apparatus into the bent configuration, in which the blood test strip stays inserted in the cartridge guide and the electric contact terminal portion of the blood test strip remains outside the sampler body of the blood sampling cartridge; and further subsequently, inserting the electric contact terminal portion into a blood analyzer for testing.

EI 20: The method of any preceding EI or any combination of the preceding EIs, wherein the second body further comprises a blood analyzer comprising comprises a counterpart electric contact terminal portion configured to electrically connect to the electric contact terminal portion of the blood test strip, wherein the counterpart electric contact terminal portion is configured to slide along the second axis of the second body, wherein the method further comprises: subsequent to causing the actuator to exert force on the sampler body, distancing the apparatus from the subject's skin; subsequently while the apparatus is still in the straight configuration, sliding the counterpart electric contact terminal portion in the first direction to electrically connect the counterpart electric contact terminal portion to the electric contact terminal portion for analyzing the blood withdrawn from underneath the subject's skin; and causing the blood analyzer to analyze the blood and provide an analysis result.

### Other Considerations

Although the implementations of the inventions have been disclosed in the context of certain implementations and examples, it will be understood by those skilled in the art that the present inventions extend beyond the specifically disclosed implementations to other alternative implementations and/or uses of the inventions and obvious modifications and equivalents thereof. In addition, while a number of variations of the inventions have been shown and described in detail, other modifications, which are within the scope of the inventions, will be readily apparent to those of skill in the art based upon this disclosure. It is also contemplated that various combinations or subcombinations of the specific features and aspects of the implementations may be made and still fall within one or more of the inventions. Accordingly, it should be understood that various features and aspects of the disclosed implementations can be combined with or substituted for one another in order to form varying modes of the disclosed inventions. Thus, it is intended that the scope of the present inventions herein disclosed should not be limited by the particular disclosed implementations described above, and that various changes in form and details may be made without departing from the spirit and scope of the present disclosure as set forth in the following claims.

1. A blood sampling cartridge comprising:
a blood test strip extending between a first end and a second end along a longitudinal axis thereof, the blood test strip comprising (i) a blood contact portion at or adjacent the first end and (ii) an electric contact terminal portion at or adjacent the second end;
a blood sampler comprising a sampler body and a needle fixed to the sampler body;
the needle comprising a distal end and a proximal end, and configured to withdraw blood from underneath a subject's skin and to cause the withdrawn blood to flow to the sampler body; and
the sampler body comprising a front end and a back end along a longitudinal axis thereof, the sampler body further comprising an inner space and a back opening formed at or toward the back end,
wherein the needle is fixed to the sampler body such that the proximal end of the needle is located inside the sampler body and in fluid communication with the inner space,
wherein the back opening configured to receive the first end of the blood test strip for inserting the first end of the blood test strip into the inner space,
wherein the sampler body further comprises a guide extending along the longitudinal axis of the sampler body and configured to guide traveling of the blood test strip in a first direction of the longitudinal axis from the back end toward the front end,
wherein the inner space further comprises a blood collection area located at or adjacent the proximal end of the needle into which blood withdrawn through the needle flows out of the proximal end of the needle,
wherein the sampler body comprises a stopper configured to stop the blood test strip from further traveling in the first direction when the blood contact portion of the blood test strip reaches the blood collection area of the inner space, at which the blood test strip is fully inserted into the inner space,
wherein the sampler body and the blood test strip are configured such that, when the blood test strip is fully inserted into the inner space, the electric contact terminal portion of the blood test strip remains exposed outside the back opening.

2. The blood sampling cartridge of claim 1, wherein the needle comprises a channel with hydrophilic surfaces configured to cause capillary action to cause the blood to flow toward the proximal end.

3. The blood sampling cartridge of claim 1, wherein the sampler body comprises at least one through hole for providing fluid communication between the inner space and outside the sampler body such that air pressure within the inner space is released through the at least one through hole as blood withdrawn through the needle flows into the inner space.

4. The blood sampling cartridge of claim 1, wherein the sampler body comprises a needle attachment piece to which a portion of the needle is fixed, a first piece and a second piece that are integrated together, wherein the needle attachment piece is received at a predetermined location between the first piece and the second piece, wherein the first piece and the second piece together define the inner space.

5. The blood sampling cartridge of claim 1, wherein the sampler body further comprises a mechanism configured to engage with a counterpart mechanism of the blood test strip for inhibiting the blood test strip from being pulled out of the inner space in a second direction opposite to the first direction after the blood test strip has been fully inserted into the inner space.

6. The blood sampling cartridge of claim 1, wherein the sampler body further comprises a mechanism configured to damage the blood test strip when the blood test strip from is pulled out of the inner space in a second direction opposite to the first direction after the blood test strip has been fully inserted into the inner space.

7. The blood sampling cartridge of claim 1, wherein the blood test strip is fully inserted into the inner space, and the blood contact portion of the blood test strip is at the blood collection area of the inner space such that blood withdrawn through the needle is to immediately contact the blood contact portion of the blood test strip.

8. A method of making the blood sampling cartridge of claim 1, the method comprising:
providing the blood test strip;
providing the blood sampler comprising the sampler body and the needle fixed to the sampler body;
inserting the first end of the blood test strip through the back opening of the sampler body; and
subsequently continuing to insert the blood test strip into the sampler body by pushing the blood test strip further into the back opening generally in the first direction until the blood test strip is stopped.

9. An apparatus for launching the blood sampling cartridge of claim 1, the apparatus comprising:
a first body elongated along a first axis and comprising:
a skin-contacting tip located at a front end of the first body along the first axis and comprising a through hole,
a rear opening located at a rear end of the first body along the first axis and configured to receive the blood sampling cartridge, and
a cartridge guide formed inside the first body and configured to guide traveling of the blood sampler of the blood sampling cartridge, subsequent to insertion via the rear opening, in a first direction of the first axis from the rear end toward the front end; and
a second body elongated along a second axis and comprising an actuator configured to exert force along the second axis,
wherein the first body and the second body are hingedly connected to hingedly move between a straight configuration and a bent configuration,
wherein the first axis and the second axis are aligned in the straight configuration, whereas the first axis and the second axis are not aligned in the bent configuration,
wherein, in the straight configuration, the second body blocks the rear opening of the first body such that the blood sampling cartridge cannot be inserted into the cartridge guide in the straight configuration,
wherein, in the bent configuration, the first axis and the second axis are not aligned, and the second body does not block the rear opening of the first body such that the blood sampling cartridge is insertable into the cartridge guide through the rear opening of the first body in the bent configuration,
wherein, upon inserting the blood sampling cartridge into the cartridge guide through the rear opening in the bent configuration, the distal end of the needle stays within the first body,
wherein, in the straight configuration with the blood sampling cartridge inserted into the cartridge guide, the actuator of the second body is configured to exert force on the sampler body of the blood sampling cartridge along the second axis to cause the blood sampling cartridge to travel in the first direction along the cartridge guide so that the distal end of the needle advances in the first direction through the through hole of the skin-contacting tip of the first body.

10. The apparatus of claim 9, wherein the second body further comprises a blood analyzer comprising a counterpart electric contact terminal portion configured to electrically connect to the electric contact terminal portion of the blood test strip.

11. The apparatus of claim 10, wherein the counterpart electric contact terminal portion is configured to slide along the second axis of the second body for engaging with the electric contact terminal portion of the blood test strip when the apparatus is in the straight configuration.

12. The apparatus of claim 9, wherein the apparatus in the straight configuration is configured for gripping with one hand, wherein the actuator comprises a push button formed on the second body for pressing with a finger of the hand and a mechanism configured to transfer force from pressing the push button to the sampler body of the blood sampling cartridge.

13. The apparatus of claim 9, wherein the actuator comprises a spring-loaded actuator comprising a spring mechanism configured to store spring energy for generating the force for exertion on the sampler body, wherein the second body further comprises a trigger configured to initiate a release of the spring energy.

14. The apparatus of claim 13, wherein the spring mechanism is configured to store the spring energy in response to the first body and the second body hingedly moving from the bent configuration to the straight configuration such that the trigger is ready to initiate the release of the spring energy upon turning to the straight configuration.

15. The apparatus of claim 9, wherein the blood sampling cartridge is inserted into the cartridge guide of the first body, wherein the electric contact terminal portion of the blood test strip remains exposed outside the back opening of the sampler body of the blood sampling cartridge.

16. A method of sampling blood, the method comprising:
providing the apparatus of claim 9, in which the blood test strip is fully inserted into the inner space;
inserting the blood sampling cartridge into the cartridge guide through the rear opening of the first body while the apparatus is in the bent configuration in which the second body does not block the rear opening of the first body, wherein, upon inserting the blood sampling cartridge into the cartridge guide through the rear opening in the bent configuration, the distal end of the needle is positioned between the rear opening and the through hole of the first body;
subsequently configuring the apparatus into the straight configuration;
holding the apparatus in the straight configuration with one hand;
positioning the skin-contacting tip at the front end of the first body against the subject's skin; and
causing the actuator to exert force on the sampler body of the blood sampling cartridge to cause the blood sampling cartridge to travel in the first direction and the distal end of the needle to advance through the through hole of the skin-contacting tip for piercing into the subject's skin, such that blood is withdrawn from underneath the subject's skin the blood flows through the needle by capillary action to the blood collection area of the inner space and reaches the blood contact portion of the blood test strip in the inner space.

17. The method of claim 16, wherein throughout the steps of the method, the electric contact terminal portion of the blood test strip remains outside the back opening of the sampler body of the blood sampling cartridge.

18. The method of claim 16, further comprising:
subsequent to causing the actuator to exert force on the sampler body, maintaining the skin-contacting tip in contact with the subject's skin for a period between one second and 10 seconds for completing the withdrawal of the blood.

19. The method of claim 16, further comprising:
subsequent to causing the actuator to exert force on the sampler body, distancing the apparatus from the subject's skin;
subsequently, reconfiguring the apparatus into the bent configuration, in which the blood test strip stays inserted in the cartridge guide and the electric contact terminal portion of the blood test strip remains outside the sampler body of the blood sampling cartridge; and
further subsequently, inserting the electric contact terminal portion into a blood analyzer for testing.

20. The method of claim 16, wherein the second body further comprises a blood analyzer comprising comprises a counterpart electric contact terminal portion configured to electrically connect to the electric contact terminal portion of the blood test strip, wherein the counterpart electric contact terminal portion is configured to slide along the second axis of the second body, wherein the method further comprises:
subsequent to causing the actuator to exert force on the sampler body, distancing the apparatus from the subject's skin;
subsequently while the apparatus is still in the straight configuration, sliding the counterpart electric contact terminal portion in the first direction to electrically connect the counterpart electric contact terminal portion to the electric contact terminal portion for analyzing the blood withdrawn from underneath the subject's skin; and
causing the blood analyzer to analyze the blood and provide an analysis result.
